(19) United States
(12) Patent Application Publication
(10) Pub. No.: US 2024/0327462 A1
(43) Pub. Date: Oct. 3, 2024

(54) SHORT PEPTIDES, PHARMACEUTICAL COMPOSITIONS COMPRISING THE SAME, AND USES THEREOF IN TREATING NEUROLOGICAL DISEASES

(71) Applicants: FU JEN CATHOLIC UNIVERSITY, New Taipei City (TW); Mackay Medical College, New Taipei City (TW)

(72) Inventors: Huai-Jen TSAI, New Taipei City (TW); Bing-Chang LEE, New Taipei City (TW); Cheng-Yung LIN, New Taipei City (TW)

(21) Appl. No.: 18/621,153

(22) Filed: Mar. 29, 2024

Related U.S. Application Data
(60) Provisional application No. 63/455,997, filed on Mar. 31, 2023.

(51) Int. Cl. C07K 7/08; A61K 38/00; A61P 25/28

(52) U.S. Cl. CPC C07K 7/08; A61P 25/28; A61K 38/00

(57) ABSTRACT

Disclosed herein is a short peptide consisting of an amino acid sequence at least 85% identical to SEQ ID NO: 1. According to some embodiments of the present disclosure, the short peptide consists of an amino acid sequence 100% identical to SEQ ID NO: 1, 2, 3 or 4. According to certain embodiments of the present disclosure, the short peptide is useful in treating neurological diseases via enhancing neurite outgrowth. Accordingly, also disclosed herein are a pharmaceutical composition comprising the short peptide, and uses thereof in treating neurological diseases.

### CROSS-REFERENCE TO RELATED APPLICATIONS

This application relates to and claims the benefit of U.S. Provisional Application No. 63/455,997, filed Mar. 31, 2023; the content of the application is incorporated herein by reference in its entirety.

### SEQUENCE LISTING XML

The present application is being filed along with a Sequence Listing XML in electronic format. The Sequence Listing XML is provided as an XML file entitled MYHP_0222US_SEQ_Listing.xml, created Mar. 29, 2024, which is 5 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to novel peptides, and uses thereof in treating neurological diseases.

### 2. Description of Related Art

Neurological disease refers to any afflictions arising from, or causing dysfunction to, the central nervous system (CNS) and/or the peripheral nervous system (PNS) of an individual. It can cause a wide ranges of symptoms, including headaches, dizziness, memory problems, vision problems, speech problems, cognitive difficulties, muscle weakness, paralysis, seizures, pain, and fainting and loss of consciousness. There are more than 600 neurological diseases. According to the primary location affected, the primary type of dysfunction involved, or the primary type of cause, neurological diseases can be categorized into genetic neurological disorders (such as muscular dystrophy and neurofibromatosis), neurodegenerative diseases (such as amyotrophic lateral sclerosis, Alzheimer's disease and Parkinson disease), disorders associated with blood vessels (such as stroke), disorder associated with immunity (such as multiple sclerosis and myasthenia gravis), brain or spinal cord injury, problems in the development of the nervous system (such as spina bifida), convulsive disorders (such as epilepsy), brain tumors, infections (such as meningitis and HIV neuropathy), etc. However, in clinical practice, there is significant overlap with many patients having mixed symptoms and medical histories, making categorization difficult.

The treatment of neurological diseases usually varies with the conditions and the severity of diseases. Unfortunately, most medicines merely improve the symptoms or slow the progression of symptoms without curing the underlying diseases. Management and treatment of neurological diseases is one of the biggest challenges facing medicine today. Accordingly, there is a continuing interest in developing a novel agent and method for treating neurological diseases.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The first aspect of the present disclosure is directed to a short peptide consisting of an amino acid sequence at least 85% identical to “WEAFARGTKALMDEVV” (SEQ ID NO: 1).

According to some embodiments of the present disclosure, the short peptide consists of an amino acid sequence 100% identical to SEQ ID NO: 1.

In certain embodiments, at least one mutation is present in the amino acid sequence of SEQ ID NO: 1.

According to one embodiment, one mutation is present in the amino acid sequence of SEQ ID NO: 1, in which the amino acid residue alanine (A) at position 10 of SEQ ID NO: 1 is substituted with amino acid residue proline (P). In this embodiment, the short peptide consists of an amino acid sequence 100% identical to “WEAFARGTKPLMDEVV” (SEQ ID NO: 2).

According to one embodiment, one mutation is present in the amino acid sequence of SEQ ID NO: 1, in which the amino acid residue lysine (K) at position 9 of SEQ ID NO: 1 is substituted with amino acid residue arginine (R). In this embodiment, the short peptide consists of an amino acid sequence 100% identical to “WEAFARGTRALMDEVV” (SEQ ID NO: 3).

According to another embodiment, two mutations are present in the amino acid sequence of SEQ ID NO: 1, in which the amino acid residue aspartic acid (D) at position 13 of SEQ ID NO: 1 is substituted with amino acid residue arginine (R), and the amino acid residue glutamic acid (E) at position 14 of SEQ ID NO: 1 is substituted with amino acid glutamine (Q). In this embodiment, the short peptide consists of an amino acid sequence 100% identical to “WEAFARGTKALMRQVV” (SEQ ID NO: 4).

Optionally, the short peptide is acetylated at its N-terminus and/or amidated at its C-terminus.

The second aspect of the present disclosure pertains to a pharmaceutical composition for treating neurological diseases. The pharmaceutical composition comprises at least one of the present short peptide (i.e., the short peptide of SEQ ID NO: 1, 2, 3 or 4), and a pharmaceutically acceptable carrier.

Also disclosed herein is a method of treating a neurological disease in a subject. The method comprises administering to the subject an effective amount of the short peptide or pharmaceutical composition of the present disclosure.

Examples of neurological disease treatable with the present method include, but are not limited to, amyotrophic lateral sclerosis (ALS), spinal muscular atrophy (SMA), Alzheimer's disease (AD), Parkinson disease (PD), Huntington's disease (HD), frontotemporal lobar dementia (FTLD), Friedreich's ataxia, age-related macular degeneration and Creutzfeldt-Jakob disease.

In general, the subject is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings.

FIG. 1 depicts the effect of the present short peptides on neurite outgrowth according to Example 1 of the present disclosure. Panel (A): Quantification of the length of neurite outgrowth. Panel (B): Expressed protein level of phosphorylated cofilin (p-cofilin). Sol8 CM: The conditioned medium (culture medium) obtained from culturing wild-type Sol8 cells. Sol-NogoA CM: The conditioned medium (culture medium) obtained from culturing NogoA-overexpressed Sol8 cells. * P<0.05; ** P<0.01; *** P<0.001.

FIG. 2 depicts the effect of the present short peptides on axonal branching of motor neurons in zebrafish embryos according to Example 2 of the present disclosure. ** P<0.01; *** P<0.001.

FIG. 3 depicts the effect of the present short peptides on delay denervation of neuromuscular junction (NMJ) in ALS mice according to Example 2 of the present disclosure. *** P<0.001.

FIG. 4 depicts the effect of the present short peptides on the improvement of moving capability (Panel A) and prolonged life-span (Panel B) of ALS mice. Panel (A): The total distance of movement averaged from ALS mice administrated with specified treatments as indicated. Panel (B): The cumulative survival rate of each group of ALS mice administered with specified treatments as indicated. * P<0.05; *** P<0.001.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “short peptide” as used herein refers to a polymer having less than 20 amino acid (a.a.) residues. Preferably, the short peptide has 5 to 20 a.a. residues in length; more preferably, 8 to 20 a.a. residues in length. According to some embodiments of the present disclosure, the short peptide has 16 a.a. residues in length. Where an a.a. sequence is provided herein, L-, D-, or beta a.a. versions of the sequence are also contemplated. Peptides also include a.a. polymers in which one or more a.a. residues are an artificial chemical analogue of a corresponding naturally occurring a.a., as well as to naturally occurring a.a. polymers. In addition, the term applies to amino acids joined by a peptide linkage or by other, “modified linkages” (e.g., where the peptide bond is replaced by an α-ester, a β-ester, a thioamide, phosphonamide, carbomate, hydroxylate, and the like).

In certain embodiments, conservative substitutions of the a.a. residues comprising any of the sequences described herein are contemplated. In various embodiments, one, two, three, four, or five different residues are substituted. The term “conservative substitution” is used to reflect a.a. substitutions that do not substantially alter the activity (e.g., biological or functional activity and/or specificity) of the molecule. Typically, conservative a.a. substitutions involve substitution one a.a. residue for another a.a. residue with similar chemical properties (e.g., charge or hydrophobicity). Certain conservative substitutions include “analog substitutions” where a standard a.a. is replaced by a non-standard (e.g., rare, synthetic, etc.) a.a. differing minimally from the parental residue. A.a. analogs are considered to be derived synthetically from the standard a.a. without sufficient change to the structure of the parent, are isomers, or are metabolite precursors.

As used herein, the term “neurological disease” refers to a neuropathy, a neurodegenerative disease and/or other neuron-associated diseases caused by mechanical damage (e.g., trauma), chemical damage (e.g., neurotoxin, immunosuppression resulting from the treatment or side-effect), immune response (e.g., inflammation or autoimmune), or infection. Exemplary neurological diseases include, but are not limited to, ALS, SMA, AD, PD, HD, FTLD, Friedreich's ataxia, age-related macular degeneration and Creutzfeldt-Jakob disease. The neuron-associated disease may include ocular disease caused by neuronal damage (e.g., glaucoma), Bell's palsy, or other forms of localized paralysis, neuron based impotence, such as caused by nerve trauma following radical prostatectomy, or other complaints. In the present disclosure, the term “neurological disease” can be any of central nervous system (CNS) disease, peripheral nervous system (PNS) disease, sympathetic nervous system disease or parasympathetic nervous system disease.

As used herein, the term “neurite outgrowth” refers to the process of axons or dendrites growing out of the cell body of a neuron. In general, the neurite outgrowth plays a key role in the development and regeneration of neurons. The neurite outgrowth improves the neural connectivity thereby promoting the synapse formation or remodeling the synapses.

“Percentage (%) sequence identity” with respect to the peptide sequences identified herein is defined as the percentage of a.a. residues in a candidate sequence that are identical with the a.a. residues in the specific peptide sequence, after aligning the sequences and introducing gaps, if necessary, to achieve the maximum percent sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two peptide sequences was carried out by computer program BLASTP (protein-protein BLAST) provided online by Nation Center for Biotechnology Information (NCBI). The percentage sequence identity of a given peptide sequence A to a given peptide sequence B (which can alternatively be phrased as a given peptide sequence A that has a certain % peptide sequence identity to a given peptide sequence B) is calculated by the formula as follows:

$$\frac{X}{Y} \times 100 \%$$

where X is the number of a.a. residues scored as identical matches by the sequence alignment program BLAST in that program's alignment of A and B, and where Y is the total number of a.a. residues in A or B, whichever is shorter.

The phrase “pharmaceutically acceptable” refers to molecular entities and compositions that are “generally regarded as safe,” e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “administered”, “administering” or “administration” are used interchangeably herein to refer a mode of delivery, including, without limitation, intravenously, intraarterially, intraperitoneally, intracerebrally or intrathecally delivering an agent (e.g., the short peptide, mutant peptide or pharmaceutical composition) of the present invention.

As used herein, the term “treat,” “treating” and “treatment” are interchangeable, and encompasses partially or completely preventing, ameliorating, mitigating and/or managing a symptom, a secondary disorder or a condition associated with the impairment of neurons. The term “treating” as used herein refers to application or administration of the short peptide, mutant peptide, or pharmaceutical composition of the present disclosure to a subject, who has a symptom, a secondary disorder or a condition associated with the impairment of neurons, with the purpose to partially or completely alleviate, ameliorate, relieve, delay onset of, inhibit progression of, reduce severity of, and/or reduce incidence of one or more symptoms, secondary disorders or features associated with the impairment of neurons. Symptoms, secondary disorders, and/or conditions associated with the impairment of neurons include, but are not limited to, headaches, dizziness, memory problems, vision problems, speech problems, cognitive difficulties, muscle weakness, paralysis, seizures, pain, and fainting and loss of consciousness. Treatment may be administered to a subject who exhibits only early signs of such symptoms, disorder, and/or condition for the purpose of decreasing the risk of developing the symptoms, secondary disorders, and/or conditions associated with the impairment of neurons. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Persons having ordinary skills could calculate the human equivalent dose (HED) for the medicament (such as the present short peptide, mutant peptide, or pharmaceutical composition) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that is treatable with the short peptide, mutant peptide, pharmaceutical composition, and/or method of the present invention. The term “subject” is intended to refer to both the male and female gender unless one gender is specifically indicated.

### II. Description of the Invention

The present disclosure is based, at least in part, on the discovery that a short peptide or its mutant is useful in enhancing neurite outgrowth, and accordingly, the short peptide or a mutant thereof may serve as a candidate for the development of a medicament for treating neurological diseases, especially the neurological diseases associated with and/or caused by the impairment of neurons.

The first aspect of the present disclosure is thus directed to a short peptide or its mutants (i.e., the present mutated peptides). According to some embodiments of the present disclosure, the short peptide designated as “FD1 peptide” consists of an a.a. sequence at least 85% (e.g., 85%, 86%, 87%, 88%, 89%, 90%, 91%, 92%, 93%, 94%, 95%, 96%, 97%, 98%, 99% or 100%) identical to “WEAFARGTKALMDEVV” (SEQ ID NO: 1, from N-terminus to C-terminus). Preferably, the FD1 peptide consists of an a.a. sequence at least 90% identical to SEQ ID NO: 1. More preferably, the FD1 peptide consists of an a.a. sequence at least 95% identical to SEQ ID NO: 1. In one specific embodiment, the FD1 peptide consists of the a.a. sequence of SEQ ID NO: 1 (i.e., having an a.a. sequence 100% identical to SEQ ID NO: 1).

According to some embodiments, the mutated peptide is designated as “FD2 peptide”, which consists of an a.a. sequence at least 85% identical to “WEAFARGTKPLMDEVV” (SEQ ID NO: 2, from N-terminus to C-terminus); preferably, at least 90% identical to SEQ ID NO: 2; more preferably, at least 95% identical to SEQ ID NO: 2. In one specific embodiment, the FD2 peptide consists of the a.a. sequence of SEQ ID NO: 2 (i.e., having an a.a. sequence 100% identical to SEQ ID NO: 2).

According to alternative embodiments, the mutated peptide is designated as “FD3 peptide”, which consists of an a.a. sequence at least 85% identical to “WEAFARGTRALMDEVV” (SEQ ID NO: 3, from N-terminus to C-terminus); preferably, at least 90% identical to SEQ ID NO: 3; more preferably, at least 95% identical to SEQ ID NO: 3. In one specific embodiment, the FD3 peptide consists of the a.a. sequence of SEQ ID NO: 3 (i.e., having an a.a. sequence 100% identical to SEQ ID NO: 3).

According to alternative embodiments, the mutated peptide is designated as “FD4 peptide”, which consists of an a.a. sequence at least 85% identical to “WEAFARGTKALMRQVV” (SEQ ID NO: 4, from N-terminus to C-terminus); preferably, at least 90% identical to SEQ ID NO: 4; more preferably, at least 95% identical to SEQ ID NO: 4. In one specific embodiment, the FD4 peptide consists of the a.a. sequence of SEQ ID NO: 4 (i.e., having an a.a. sequence 100% identical to SEQ ID NO: 4).

Optionally, the present short peptide may be modified at its N-terminus or C-terminus. Examples of N-terminal modifications include, but are not limited to, N-glycated, N-alkylated, and N-acetylated a.a.. A terminal modification can include a pegylation. An example of C-terminal modification is a C-terminal amidated a.a.. Alternatively, one or more peptide bond may be replaced by a non-peptidyl linkage, the individual a.a. moieties may be modified through treatment with agents capable of reacting with selected side chains or terminal residues.

Various functional groups may also be added at various points of the present short peptides that are susceptible to chemical modification. Functional groups may be added to the termini of the short peptide. In some embodiments, the function groups improve the activity of the short peptide with regard to one or more characteristics, such as improving the stability, efficacy, or selectivity of the short peptide; improving the penetration of the short peptide across cellular membranes and/or tissue barrier; reducing toxicity or clearance; and improving resistance to expulsion by cellular pump and the like. Non-limited examples of suitable functional groups are those that facilitate transport of a peptide attached thereto into a cell, for example, by reducing the hydrophilicity and increasing the lipophilicity of the peptide, these functional groups may optionally and preferably be cleaved in vivo, either by hydrolysis or enzymatically, inside the cell. Hydroxy protecting groups include esters, carbonates and carbamate protecting groups. Amine protecting groups include alkoxy and aryloxy carbonyl groups. Carboxylic acid protecting groups include aliphatic, benzylic and aryl esters.

According to some embodiments of the present disclosure, each of the short peptide (i.e., FD1 peptide) and the mutated peptides (i.e., FD2, FD3 and FD4 peptides) is useful in enhancing the outgrowth of the neurite of a neuron. The second aspect of the present disclosure thus pertains to a pharmaceutical composition or medicament for treating neurological diseases associated with and/or caused by the impairment of neurons. The present pharmaceutical composition or medicament comprises the present short peptide (i.e., FD1 peptide) or mutated peptide (i.e., FD2, FD3 or FD4 peptide), and a pharmaceutically acceptable carrier.

Depending on desired purposes, the pharmaceutically acceptable carrier may be a liposome, nanoparticle, diluent, dispersion medium, buffer, stabilizing agent, or any solutions or substances compatible with pharmaceutical administration.

The instant peptide (i.e., the short peptide or mutated peptide) is present at a level of about 0.1% to 99% by weight, based on the total weight of the pharmaceutical composition or medicament. In some embodiments, the instant peptide is present at a level of at least 1% by weight, based on the total weight of the pharmaceutical composition or medicament. In certain embodiments, the instant peptide is present at a level of at least 5% by weight, based on the total weight of the pharmaceutical composition or medicament. In still other embodiments, the instant peptide is present at a level of at least 10% by weight, based on the total weight of the pharmaceutical composition or medicament. In still yet other embodiments, the instant peptide is present at a level of at least 25% by weight, based on the total weight of the pharmaceutical composition or medicament.

In some embodiments, the pharmaceutical composition or medicament is formulated in liquid dosage form suitable for parenteral (e.g., intravenous, intraarterial, intracerebral, or intrathecal) administration. To this purpose, the pharmaceutical composition or medicament may be formulated as an isotonic suspension, solution or emulsion in oily or aqueous vehicles, and may contain formulary agents such as suspending, stabilizing or dispersing agents. The preparation of such liquid dosage form, having due regard to pH, isotonicity, stability, and the like, is within the skill in the art.

A preferred pharmaceutical composition or medicament for parenteral injection should contain, in addition to the present peptide, an isotonic vehicle such as sodium chloride injection, Ringer's injection, dextrose injection, dextrose and sodium chloride injection, lactated Ringer's injection, or other vehicle as known in the art. The pharmaceutical composition of the invention may also contain stabilizers, preservatives, buffers, antioxidants, or other additives known to those of skill in the art.

The third aspect of the present disclosure provides a method of treating a neurological disease in a subject. The method comprises administering to the subject an effective amount of a short peptide (i.e., FD1 peptide), a mutated peptide (i.e., FD2, FD3 or FD4 peptide), or a pharmaceutical composition in accordance with any embodiments of the present disclosure.

Examples of neurological disease treatable by the present method include, but are not limited to, ALS, SMA, AD, PD, HD, FTLD, Friedreich's ataxia, age-related macular degeneration and Creutzfeldt-Jakob disease.

Also disclosed herein is a method of enhancing the outgrowth of the neurite of a neuron in a subject. The method comprises administering to the subject an effective amount of the short peptide (i.e., FD1 peptide), mutated peptide (i.e., FD2, FD3 and FD4 peptide), or pharmaceutical composition in accordance with any embodiments of the present disclosure.

The subject treatable by the present method is a mammal; for example, a human, a mouse, a rat, a hamster, a guinea pig, a rabbit, a dog, a cat, a cow, a goat, a sheep, a monkey, and a horse. Preferably, the subject is a human.

According to some embodiments, the subject is afflicted with a neurological disease. In these embodiments, the neurological disease is caused by the impairment of neurites; for example, the subject may suffer from a neuronal damage or neuropathy caused by a mechanical damage (e.g., trauma), a chemical damage (e.g., neurotoxin, immunosuppression resulting from the treatment or the side-effect of the treatment), and/or a biological damage (e.g., infection, inflammation, autoimmune, aging, disease, metabolic disorder or abnormal expression of protein) that interferes the transduction of electrochemical stimulation in the subject. The present method is useful in enhancing the outgrowth of the neurites in the subject so as to treat the neurological disease.

Depending on desired purposes, the present short peptide, mutant peptide or pharmaceutical composition may be administered to the subject via any suitable routes, for example, intravenous, intraarterial, intraperitoneal, intracerebral or intrathecal injection.

According to some embodiments, the subject is a mouse, in which the present short peptide, mutated peptide or pharmaceutical composition is administered to the subject in the amount of about 0.1 mg/Kg to 1,000 mg/Kg body weight per dose; for example, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420,430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 or 1,000 mg/Kg body weight per dose. Preferably, the effective amount is about 1 mg/Kg to 100 mg/Kg. More preferably, the effective amount is about 5 mg/Kg to 50 mg/Kg. According to one working example, about 10 mg/Kg of the present peptide, mutant peptide or pharmaceutical composition are sufficient to produce a therapeutic effect (e.g., enhance the outgrowth of the neurites and/or treat the neurological disease) in the subject.

A skilled artisan could calculate the human equivalent dose (HED) of the present short peptide, mutant peptide or pharmaceutical composition, based on the doses determined from animal models. Accordingly, the effective HED of the present short peptide, mutant peptide or pharmaceutical composition is about 10 μg/Kg to 100 mg/Kg body weight per dose for human; in other words, the effective HED of the present short peptide, mutant peptide or pharmaceutical composition may be any of, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980 or 990 μg/Kg, or 1, 2, 3, 4, 5, 6, 7, 8, 9, 10, 20, 30, 40, 50, 60, 70, 80, 90 or 100 mg/Kg body weight per dose for human.

Depending on the particular condition (e.g., the physical condition of the patient, the type of neurological disease, and the severity of the neurological disease), the effective amount of the present short peptide, mutant peptide or pharmaceutical composition may be divided into one or more doses throughout a designated time period so as to enhance the outgrowth of the neurites and/or treat the neurological disease in the subject; for example, the effective amount of the present short peptide, mutant peptide or pharmaceutical composition may be divided into 1, 2, 3, 4, 5, 6, 7, 8, 9, 10 or more doses. The time period between two consecutive doses may be 1, 2, 3, 4, 5, 6, 7, 8, 9, 10 or more days. The practitioner may adjust the administration procedure in accordance with the desired effects.

As could be appreciated, the present method can be applied to the subject, alone or in combination with an additional therapy (e.g., a neuroprotective or neurotherapeutic agent) that has some beneficial effects on the prevention or treatment of neurological diseases. Depending on the intended/therapeutic purpose, the present method can be applied to the subject before, during, or after the administration of the additional therapy.

The present disclosure also provides uses of the present short peptide (i.e., FD1 peptide) or its mutant (i.e., FD2, FD3 or FD4 peptide) for the manufacture of a medicament for use in the treatment of a neurological disease (e.g., ALS, SMA, AD, PD, HD, FTLD, Friedreich's ataxia, age-related macular degeneration, Creutzfeldt-Jakob disease, or a combination thereof).

Also disclosed herein is the present short peptide (i.e., FD1 peptide) or its mutant (i.e., FD2, FD3 or FD4 peptide) for use in the treatment of a neurological disease (e.g., ALS, SMA, AD, PD, HD, FTLD, Friedreich's ataxia, age-related macular degeneration, Creutzfeldt-Jakob disease, or a combination thereof).

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

### Materials and Methods

### Peptide Synthesis and Preparation

Four peptides were provided in the study, including a short peptide derived from human PGK1, and three mutants of the short peptide. The peptides were produced by Fmoc solid-phase synthesis, which started at the carboxyl terminus (C-terminus) of the peptide and proceeded toward the amino terminus (N-terminus) of the peptide.

The thus-produced peptides were purified by column, and were respectively designated as “FD1 peptide”, “FD2 peptide”, “FD3 peptide”, and “FD4 peptide”. The a.a. sequences of the peptides are summarized in Table 1 below.

TABLE 1
Amino acid sequences of FD1,
FD2, FD3 and FD4 peptides

| Name | Amino acid sequence* | SEQ ID NO |
|---|---|---|
| FD1 | WEAFARGTKALMDEVV | 1 |
| FD2 | WEAFARGTKPLMDEVV | 2 |
| FD3 | WEAFARGTRALMDEVV | 3 |
| FD4 | WEAFARGTKALMRQVV | 4 |
| 16 a.a | MSLSNKLTLDKLDVKG | 5 |
| (control | | |
| peptide) | | |

*The mutated amino acid residue(s) was/were marked in boldface.

### Preparation of Nogo A-Overexpressed Sol8 Cells (Sol8-NogoA Cells)

A doxycycline inducible lentiviral plasmid (pAS4.1w.Ppuro-aOn) carrying human Nogo A gene was co-transfected with Gag-expressing plasmid (pCMVΔR8.91) and VSV-G-expressing plasmid (pMD.G) into HEK-293T via LIPOFECTAMINE® 2000. After 24-48 hours, the supernatant containing viral particles was collected and centrifuged at a speed of 1,250 rpm, followed by adding to Sol8 cells (ATCC, CRL-2174) pretreated with 8 μg/ml POLYBRENE®. 24 hour later, the fresh DMEM medium containing 10% fetal bovine serum (FBS), 100 units/ml penicillin, 1% streptomycin and 4 ug/ml puromycin was added to the Sol8 cells so as to obtain NogoA-overexpressed Sol8 cells.

Preparation of Conditioned Medium (CM) from Culturing Wild-Type Sol8 Cells (Sol8 CM) or NogoA-Overexpressed Sol8 Cells (Sol8-NogoA CM)

The wild-type Sol8 cells and NogoA-overexpressed Sol8 cells (Sol8-NogoA cells) were respectively cultured and differentiated in DMEM medium containing 10% FBS, 100 units/ml penicillin and 1% streptomycin. Two days later, 1 μg/μl of doxycycline was added to the medium so as to induce the expression of target genes. The culture medium was collected and the fresh medium containing doxycycline was added to the Sol8-NogoA cells every 24 hours. The collected culture medium was then centrifuged at a speed of 3,000 rpm for 5 minutes at room temperature, and the supernatant was harvested therefrom served as CM for the following assays.

### Treatment of NSC34 Cells

NSC34 cells (mouse Neuroblastoma x Spinal Cord-34 cell line) (RRID:CVCL_D356) were cultured in DMEM medium containing 10% FBS, 100 units/ml penicillin and 1% streptomycin. To investigate the effect of the present peptides (including FD1, FD2, FD3 and FD4) on neurons, NSC34 cells were cultivated in differentiation medium (DMEM medium containing 2.5% FBS, 100 units/ml penicillin and 1% streptomycin) for 24 hours, and then continued cultivating in CM obtained from culturing Sol8-NogoA cells (Sol8-NogoA CM) with or without addition of the present peptide (i.e., FD1, FD2, FD3 or FD4) in an amount of 66 ng/ml. The CM with or without containing the present peptide was refreshed daily. Cells were fixed with paraformaldehyde (PFA) for 48 hours, followed by measuring the length of neurite outgrowth using software based the neurites shown on microscopic image. Each data was averaged from three independent experiments and the length presented in each time was averaged from more than 100 counting cells. The effect of the present peptide on improving neurite outgrowth was determined by comparing the neurite length of NSC34 cells treated with the present peptide in Sol8-NogoA CM with that of NSC34 cells treated with phosphate buffered saline (PBS; serving as a control group) in Sol8-NogoA CM.

### Western Blot Analysis

The total proteins extracted from treated NSC34 cells were analyzed by sodium dodecyl-sulfate polyacrylamide gel electrophoresis (SDS-PAGE) on a 12% polyacrylamide gel. After electrophoresis, western blotting was performed. The protein levels of phosphorylated cofilin (p-cofilin) and alpha-tubulin contained in NSC34 cells cultured in medium with addition of either PBS (served as control) or the present peptide (i.e., FD1, FD2, FD3 or FD4) were determined using antibodies specifically against p-cofilin and alpha-tubulin, respectively. The intensity shown on the blot was quantified by software. Each data was averaged from three independent experiments. The relative intensity among groups was based on the comparison with that of control group set as 1.

### Transgenic Zebrafish

The transgenic line of zebrafish Tg(mnxl:GFP) which exhibits GFP-tagged motor neurons was purchased from the Zebrafish International Resource Center (ZIRC, USA). The branched primary motor neurons (PMN) derived from caudal primary (CaP) motoneurons located at somites from 11^{th} to 20^{th} of Tg(mnxl:GFP) zebrafish embryos at 30 hours post fertilization (hpf) was examined. The untreated embryos served as control group, the embryos injected with an arbitrary non-functional 16 a.a. served as mock control group, while experimental groups were embryos injected with FD1, FD2, FD3 or FD4. After injection, the percentage of zebrafish embryos exhibiting branched PMN among 20 examined embryos was quantified. Each data was averaged from three independent experiments.

The PBS and present peptides (i.e., FD1, FD2, FD3 and FD4) were respectively microinjected into the brain chamber of Tg (mnxl:GFP) embryos at 20 hpf. After 10 hours, the control embryos, mock control embryos and experimental embryos were monitored by anatomic microscope equipped with fluorescence system and digital camera.

### Mice Study

Transgenic mice of the congenic strain ALS SOD1^{G93A} carrying human SOD1 with the pathogenic G93A mutation (a mouse model of ALS that developed stereotyped phenotypes resembling the symptoms in ALS patients) were purchased from The Jackson Laboratory (JAX). The present peptide (i.e., FD1, FD2, FD3 or FD4) was initiated to inject intravenously in an amount of 250 ug (about 10 mg/Kg) into 60-day-old ALS SOD1^{G93A} mice. Injection was performed weekly. Injection of PBS served as a control group. The treated mice were monitored daily and analyzed via video tracking software.

To study the denervation of NMJ, the gastrocnemius muscles were extracted from 75-day-old ALS SOD1^{G93A} mice received with either PBS (control) or the present peptide (FD1, FD2, FD3 or FD4) and analyzed by immunostaining. In brief, the gastrocnemius muscles fixed with paraformaldehyde (PFA) were mounted on glass slides and treated with 2% TRITON™ for one hour. After blocking with PBS containing 5% bovine serum albumin (BSA) for 2 hours, the antibody against either synaptic vesicle protein (synapsin) or alpha-Bungarotoxin (α-BTX) was added to the slides, followed by incubating at 4° C. for 16 hours. The slides were washed with PBS containing 2% TRITON™ for three times, and then Cy2-conjugated anti-rabbit antibody and ALEXA FUOR™ 647-conjugated anti-mouse antibody were respectively added to the slides, followed by incubating at 4° C. for 16 hours. The slides were washed with PBS containing 2% TRITON™ for three times. The axon terminal of motoneurons via synapsin labeled by green fluorescent signal, while detected the acetylcholine receptor on motor endplates via α-BTX labeled by red fluorescent signal. The immunoblot was observed using chromogen system. The percentage of yellow signal (green signal overlapped with red signal) among total red signal shown on the 600× magnified image area (400×400 pixels) of muscle sample from PBS- and peptide-injected SOD1-G93A ALS mouse was calculated using software. Each experiment was calculated from 13-15 images.

The moving capability of ALS model mice (SOD1 G93A) injected with PBS or the present peptide (i.e., FD1, FD2, FD3 or FD4) was determined via measuring the moving trajectory of each treated mouse at 124 days after birth.

### Cumulative Survival Rate

The initial total number of ALS mice in each group (7 and 5 samples for control and FD-treated groups, respectively) was set as a 100% survival rate. During experimental period, once the ALS mouse was dead, the number and its survival days were recorded, and the cumulative survival rate was counted. When all ALS mice within the experimental group were dead, the cumulative survival rate was considered as 0%. In case that either the weight loss of a mouse was greater than 20% of original or a mouse that was not capable to eat and drink normally, euthanasia was performed and considered it as a non-survival sample. The survival days for each group were averaged from seven samples for PBS-injected control group and five samples for FD-injected experimental groups.

### Statistical Analysis

Data from at least three independent experiments under identical conditions were expressed as the mean± standard error of the mean (SEM). Student's t-test was used to analyze the differences between groups. Statistical analyses were performed using software. Statistical probability (p) was expressed as * p<0.05, ** p<0.01, and *** p<0.001.

### Example 1. The Present Peptides Enhanced In Vitro Neurite Outgrowth

The effect of the present peptides (FD1, FD2, FD3 and FD4) on neurite outgrowth was examined in this example. As described in Materials and Methods, the motor neural cells NSC34 were treated with conditioned medium (CM) collected from skeletal muscle cells Sol8 (i.e., wild-type strain (Sol8) or NogoA-overexpressed strain (Sol8-NogoA)), in the absence or presence of the present peptide (i.e., FD1, FD2, FD3 or FD4), for 48 hours.

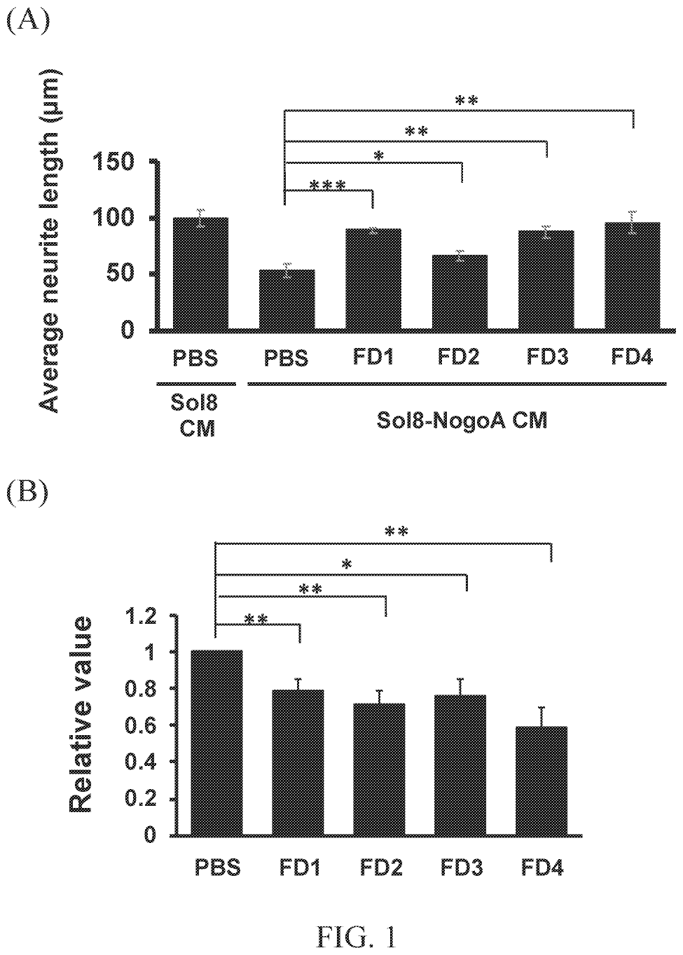

The data of FIG. 1 demonstrated that extracellular addition of the present peptide in CM was able to improve the neurite outgrowth of motor neuron cells. Compared to the average neurite length (99.4 μm) of NSC34 cells cultured in the Sol8 CM containing PBS (serving as control group), the neurite length of NSC34 cells cultured in the Sol8-NogoA CM containing PBS was reduced down to 53.6 μm (Panel (A) of FIG. 1). However, the neurite length of NSC34 cells cultured in the Sol8-NogoA CM with addition of the peptide FD1, FD2, FD3 or FD4 was significantly improved by 89.2-, 66.6-, 88.2- and 95.8-μm (Panel (A) of FIG. 1). The results demonstrated that extracellular addition of each peptide (i.e., FD1, FD2, FD3 or FD4) was capable of improving the neurite outgrowth of motor neuron cells.

Then, the expression levels of phosphorylated cofilin (p-cofilin) protein, a growth cone collapse marker, and alpha-tubulin served as internal loading control were detected. The p-cofilin level of NSC34 cells cultured in the NogoA-overexpressed Sol8 CM containing PBS (serving as control group) was normalized as 1. Based on the constant intensity of alpha-tubulin internal control employed, the p-cofilin protein level of NSC34 cells cultured in the NogoA-overexpressed Sol8 CM containing the present peptide (i.e., FD1, FD2, FD3 or FD4) exhibited a significantly reduced level of p-cofilin as compared to that of PBS control (Panel (B) of FIG. 1). This evidence suggested that decreased p-cofilin mediated by each administrated peptide (i.e., FD1, FD2, FD3 or FD4) was favorable for better neurite outgrowth as depicted in Panel (A) of FIG. 1.

The data of the example demonstrated that compared to the treatment of Sol8-NogoA CM, which inhibited neurite outgrowth via increasing the phosphorylation level of cofilin, the neurite outgrowth of NSC34 cultured in Sol8-NogoA CM but added the present peptide (i.e., FD1, FD2, FD3 or FD4) significantly increased. Accordingly, in vitro study demonstrated that each of the present peptides (including FD1, FD2, FD3 and FD4) was effective to enhance the neurite outgrowth of motor neurons.

### Example 2. The Present Peptides Enhanced In Vivo Neurite Outgrowth

Two animal models, including zebrafish model and mouse ALS model, were used in the present disclosure to evaluate the in vivo effect of the present peptides on increased axonal branching of motor neurons in zebrafish embryos and delay denervation of neuromuscular junction (NMJ) in mouse ALS model.

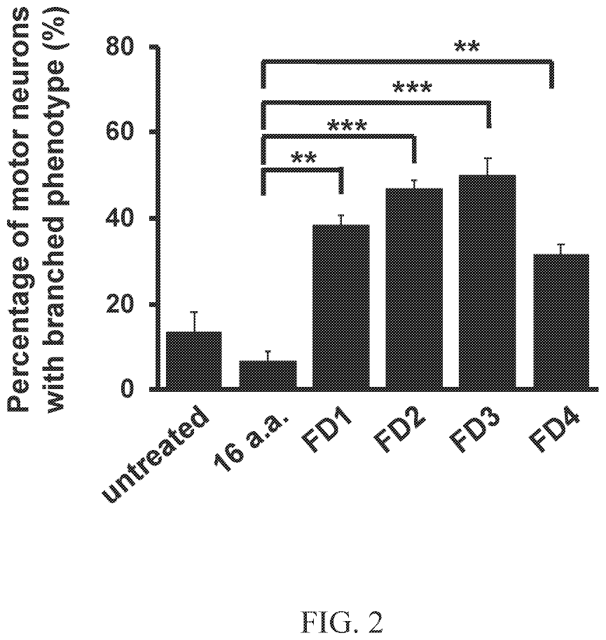

The data of FIG. 2 demonstrated that extracellular addition of the present peptide increased axonal branching of motor neurons in zebrafish embryos. The number of branched primary motor neurons (PMN) was examined in the zebrafish embryos in vivo. Quantification of the percentage of zebrafish embryos exhibiting branched PMN among 20 embryos was counted. Compared to the untreated and the 16-a.a.-injected (an arbitrary non-function peptide having the a.a. sequence of “MSLSNKLTLDKLDVKG”; SEQ ID NO: 5) control groups, the percentage of FD1-, FD2-, FD3- or FD4-peptide-injected zebrafish embryos exhibiting branched motor neurons was significantly increased (FIG. 2). This evidence demonstrated that extracellular administration of each peptide (i.e., FD1, FD2, FD3 or FD4) was able to increase axonal branching of motor neurons in zebrafish embryos.

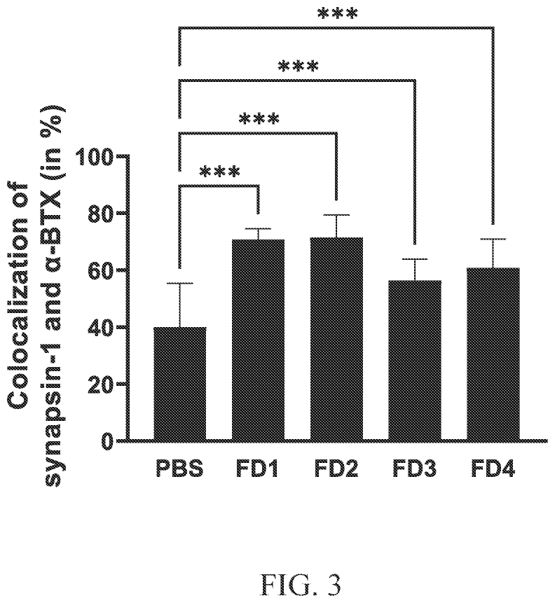

To examine the integrity of NMJ, the axonal termini of motor neurons were detected via synapsin labeled by green fluorescent signal, while the acetylcholine receptor on motor endplates was detected via α-BTX labeled by red fluorescent signal. The percentages of yellow signal (green overlapped with red signals) among total red signal presented on the muscle samples dissected from PBS- and peptide-injected SOD1-G93A ALS mouse were calculated. According to the results, the percentage of co-localized synapsin-1 and α-BTX 1 of each peptide-injected SOD1-G93A ALS mouse was significantly higher than that of PBS-injected mouse (FIG. 3). This evidence demonstrated that the injection of the present peptide (i.e., FD1, FD2, FD3 or FD4) enabled SOD1-G93A ALS to maintain the integrity of NMJ longer than that of control mice and caused the delay of NMJ denervation.

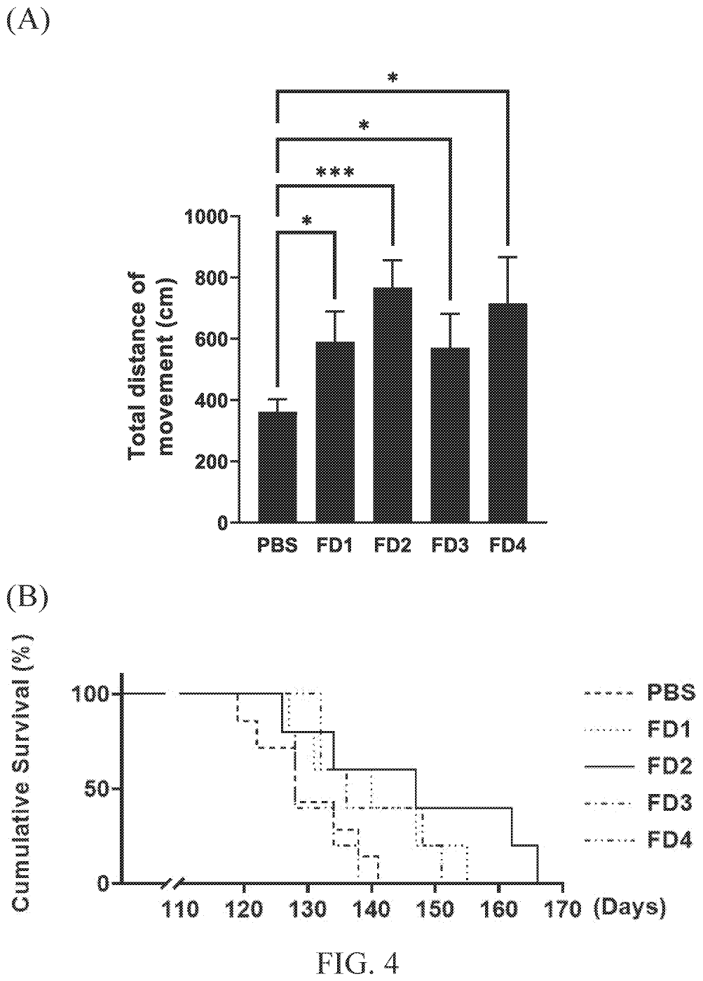

The moving capability of the mouse administered with PBS or the present peptide was measured via recording its moving trajectory. The results demonstrated that the moving distance of either FD1-, FD2- FD3- or FD4-injected ALS mice was significantly improved as compared to that of PBS-injected control ALS mice (Panel (A) of FIG. 4). The average survival days for PBS-, FD1-, FD2-, FD3- and FD4-injected ALS mice were 130.0, 140.0, 147.0, 130.8 and 139.0, respectively (Panel (B) of FIG. 4). These results revealed that the cumulated survival rate of ALS mice injected with peptide either FD1, FD2 or FD4 (5 samples of each group) exhibited a longer survival time as compared to that of PBS-injected control mice (7 samples), although the cumulated survival rate of FD3-injected ALS mice was not shown difference from that of control mice. This evidence demonstrated that intravenous injection of peptide FD1, FD2 or FD4 enables ALS mice to significantly prolong their life-span.

The data of the example demonstrated that compared to the untreated and an arbitrary non-function 16 a.a. (MSLSNKLTLDKLDVKG; SEQ ID NO: 5) control groups, the administration of the present peptide (i.e., FD1, FD2, FD3 or FD4 peptide) significantly enhanced motor neuron axonal branching in zebrafishes (FIG. 2). In the mouse model, denervation of the neuromuscular junction (NMJ) occurred in ALS mice, and the intravenously injection of the present peptide (i.e., FD1, FD2, FD3 or FD4 peptide) significantly reduced motor neuron denervation of ALS mice (FIG. 3). Furthermore, compared to the PBS control group, the present peptide (i.e., FD1, FD2, FD3 or FD4 peptide) improved motor function and prolonged survival of ALS mice (FIG. 4).

In conclusion, the present disclosure provides novel peptides, including a short peptide (i.e., FD1 peptide) and its mutants (i.e., FD2, FD3 and FD4 peptides), each of which is capable of enhancing neurite outgrowth/branching and reduced denervation of neurons; accordingly, each of the present peptides may serve as a potential candidate for the development of a medicament for treating neurological diseases.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A short peptide consisting of an amino acid sequence at least 85% identical to “WEAFARGTKALMDEVV” (SEQ ID NO: 1).

2. The short peptide of claim 1, wherein the short peptide consists of an amino acid sequence 100% identical to “WEAFARGTKALMDEVV” (SEQ ID NO: 1).

3. The short peptide of claim 1, wherein the short peptide is acetylated at its N-terminus and amidated at its C-terminus.

4. The short peptide of claim 1, wherein at least one mutation is present in the amino acid sequence of SEQ ID NO: 1.

5. The short peptide of claim 4, wherein the short peptide consists of an amino acid sequence 100% identical to “WEAFARGTKPLMDEVV” (SEQ ID NO: 2), “WEAFARGTRALMDEVV” (SEQ ID NO: 3), or “WEAFARGTKALMRQVV” (SEQ ID NO: 4).

6. A pharmaceutical composition comprising the short peptide of claim 1, and a pharmaceutically acceptable carrier.

7. A method of treating a neurological disease in a subject, comprising administering to the subject an effective amount of the short peptide of claim 1, or the pharmaceutical composition of claim 6.

8. The method of claim 7, wherein the neurological disease is amyotrophic lateral sclerosis (ALS), spinal muscular atrophy (SMA), Alzheimer's disease (AD), Parkinson disease (PD), Huntington's disease (HD), frontotemporal lober dementia (FTLD), Friedreich's ataxia, age-related macular degeneration or Creutzfeldt-Jakob disease.

9. The method of claim 7, wherein the subject is a human.


SEQUENCE LISTING (5 sequences)

SEQ ID NO: 1 - protein, 16 aa, synthetic construct
WEAFARGTKALMDEVV

SEQ ID NO: 2 - protein, 16 aa, synthetic construct
WEAFARGTKPLMDEVV

SEQ ID NO: 3 - protein, 16 aa, synthetic construct
WEAFARGTRALMDEVV

SEQ ID NO: 4 - protein, 16 aa, synthetic construct
WEAFARGTKALMRQVV

SEQ ID NO: 5 - protein, 16 aa, synthetic construct
MSLSNKLTLDKLDVKG